(19) United States
(12) Patent Application Publication
(10) Pub. No.: US 2007/0179352 A1
(43) Pub. Date: Aug. 2, 2007

(54) Device for displaying data relevant for a diabetic patient

(75) Inventors: Jette Randlov, Vaerlose (DK); Hans Henrik Thodberg, Holte (DK); Ulrik Poulsen, Hillerod (DK); Simon Lawton, Frederiksberg (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/527,074

(22) Filed: Sep. 26, 2006

Related U.S. Application Data
(63) Continuation of application No. PCT/DK05/00145, filed on Mar. 3, 2005.

(30) Foreign Application Priority Data
Mar. 26, 2004 (DK) PA 2004 00488
Dec. 9, 2004 (DK) PA 2004 01909

(51) Int. Cl. A61B 5/00; G06T 11/20

(52) U.S. Cl. 600/300; 128/921; 600/365; 345/440

(57) ABSTRACT

This invention relates to a device comprising a display for displaying graphics, text and/or combinations thereof, a processor (230) that is interfaced with said display, wherein the processor is configured to cause the display to display in a diagram, which diagram comprises a time axis indicating time relative to a habitual meal of a diabetic patient and a second axis on which units of blood glucose values are indicated, the following items: a) an indication at the point of time of the habitual meal, b) a mean or a median value of pre meal blood glucose values and/or d) a set of pre meal blood glucose measurements, and c) a mean or a median value of post meal blood glucose values, and/or e) a set of post meal blood glucose measurements. It is an advantage that said displayed items can be used as a dialogue tool between the diabetic patient and a health care personal. Furthermore, said displayed items on the device can be used as an aid in self management of diabetes. Said device can be a drug administration device or a blood glucose measuring device.

### CROSS REFERENCE TO RELATED APPLICATIONS

This application is a continuation of International Application Number PCT/DK2005/000145, filed Mar. 3, 2005, which claims priority to Danish Patent Application Numbers PA 2004 00488, filed Mar. 26, 2004 and PA 2004 01909, filed Dec. 9, 2004 and US Provisional Application No. 60/641,251 filed Jan. 4, 2005, the contents of each of which is incorporated herein in its entirety.

### FIELD OF THE INVENTION

The present invention relates to the field of health management and in particular, self-medication and treatment. More particularly the invention relates to a device capable of displaying data relevant for a diabetic patient.

### BACKGROUND OF THE INVENTION

Health problems in humans can broadly be clubbed under two categories i.e. acute and chronic. Acute diseases are sudden problems in the body that have a well-defined method for treatment and once treated; the patient is back to his normal life.

Chronic diseases on the other hand are problems that are faced by a person because of some metabolic dysfunctions. These kinds of problems are difficult to treat and require a kind of control. This control apart from regular medication and other health care regime also requires a life style management from the patient.

Diabetes is one such kind of chronic disease that requires continuing medical care and patient self-management education so as to avoid complications. Diabetes is also classified as a chronic disease. Lack of insulin (produced by pancreas) in the body results in a rise in the blood sugar level, which in turn has various effects such as excessive thirst, frequent urination, weakness and excess of ketones in the bloodstream.

People with type 1 diabetes and many people with type 2 diabetes or Gestational diabetes (Gestational diabetes occurs when a woman's body cannot make the amount of insulin needed during pregnancy) administer insulin as part of their diabetes treatment plans.

Two main kinds of insulin used in diabetes treatment are bolus insulin and background insulin, the latter is also referred to as basal insulin. Bolus insulin supplies a burst of insulin and is usually taken before or in relation to a meal. The two types of bolus insulin are rapid-acting and short-acting. Rapid-acting bolus insulin works quickly and leaves the body quickly. Short-acting bolus insulin stays in the body longer.

Basal insulin or background insulin supplies a low level of insulin throughout the day and overnight. The three kinds of background insulin are intermediate-acting, prolonged intermediate-acting and long-acting. Of the three different background insulin, long-acting insulin stays in the body the longest.

In order to keep the blood sugar level in check, diabetics administer doses of insulin at regular intervals of time. However this is not a cure but just a part of the treatment. A diabetic Health Management program would typically involve other elements such as regular exercise, food intake monitoring etc. A balance between the food intake and exercising etc has to be maintained so as to make the body behave as close as possible to a normal body.

Increased awareness in the field of diabetes has resulted in better medicines and ways for easy self-administration of insulin. The importance of health management has been realized and coordinated teams and peer group networks have been set up. In order to keep track as to how well a patient is leading his life and controlling the various parameters that would help him lead a better and healthy life, usually the patient maintains a logbook or diabetes diary with so called diary data. This logbook records various parameters such as food intake (i.e. Carbohydrates that form sugar in body), exercise, blood glucose level, insulin ejected etc. These parameters are maintained and analysed by the patient and his health care team to detect any unwanted habit or undesired deviation in the vital data.

With the advent of technology, these diaries became computerized and the patients started maintaining data on a computing device such as a Personal Computer, laptop, hand held devices etc. With the introduction of smart software and computing devices, the patient was even able to automatically analyse his data, generate reports and set reminders for various activities.

Advancement in communication technology and emergence of convergence led to interconnection of all these devices. The result was highly positive that now the patient had access to his data and reminder system at all times. The patient's data was also easily accessible to his doctor and health management team.

The breakthrough in disease self-management especially in diabetes came with the introduction of portable self-operated drug administration devices. These devices are not only easy to use but also safe. For example devices to inject insulin (for diabetes patients), inhalers (for asthma patients or diabetes patients as well), blood sample collection device, e.g. blood glucose meter, etc are widely available in the market. These devices have the dual purpose of administering the drug dosage to the patient as well as they can have advanced functionality inbuilt such as recording of patient's data to establish diary data and setting reminders for him. The device can have an alarm system as well as a display means for analyzing of the recorded data or they can transfer the data through some communication channel to an external computing device with better processing capabilities and/or bigger display means. International Publication Nos'. WO 00/32088, WO 03/005891 and WO 03/015838 all describe such medical devices, networks and method of their operation along with some of the possibilities in the domain. These publications are incorporated herein in entity by way of reference.

Various statistical means have been adopted to display the patient data for easy understanding as well as accurate and beneficial analysis. For instance, there can be a report which would show the patient's blood glucose level at various times of the day and indicate any undesired highs or lows. Similarly there can be a report for patient's food intake. These reports can be textual or in various graphical representations, such as bar graph, pie chart, histograms etc can be used to facilitate easier understanding of the results.

One such useful report is the modal day report. In this kind of display, data for several days are displayed versus the time of the day, thus superimposing many days, which allows the user to spot patterns in the data. The modal day is discussed in a co-pending Danish patent application PA 2004 01040.

The modal day view can be displayed for several types of diary data such as:
1. Blood glucose (concentration, mmol/l or mM)
2. Insulin bolus insulin administrations (IU or Insulin Units)
3. Meal (amount of glucose/meal size)
4. Exercise (intensity and duration).

This daily trend plot helps in glycaemic control vis a vis the daily activities of the patient.

In a modal day plot, a user (patient/analyst/doctor) can select the period range i.e. day, week, month, quarter, year etc. for data points to be analyzed. A target/desirable range can be decided and the analysis of data points can be done keeping those points into consideration. The software can also generate a statistical summary report.

Software like these are well known in the art and are widely available. DIABASS mobil for Palm-top, MiniMed's MMT-7311, SAS Insight are some of the examples of the packages that have aforementioned features of recording, analyzing, generating alarms etc.

However the display of data as offered by these devices/software have the drawback that the user cannot see how the blood glucose values relate to meals, especially since ingestion of meals can take place at rather different and various point of times from day to day.

The present invention overcomes this drawback since said device comprises
a display for displaying graphics, text and/or combinations thereof,
a processor that is interfaced with said display,
where said processor is configured to cause the display to display in a diagram, which diagram comprises a time axis indicating time relative to a habitual meal of a diabetic patient and a second axis on which units of blood glucose values are indicated, the following items:
a) an indication at the point of time of the habitual meal, e.g. the indication could be a line, e.g. a vertical line, a symbol, or a pictogram, e.g. a knife and fork, a cup or a glass.
b) a mean or a median value of pre meal blood glucose values, typically displayed in [mmol/l]
and/or d) a set of pre meal blood glucose measurements,
and
c) a mean or a median value of post meal blood glucose values, also typically displayed in [mmol/l] and/or e) a set of post meal blood glucose measurements.

Apart from displaying a) said device can display, including the display of a), said items in the following nine embodiments of the invention:
1) d) and c), i.e. a set of pre meal blood glucose measurements and a mean or a median value of post meal blood glucose values,
2) b) and e), i.e. a mean or a median value of pre meal blood glucose values and a set of post meal blood glucose measurements,
3) b) and c), i.e. a mean or a median value of pre meal blood glucose values and a mean or a median value of post meal blood glucose values,
4) d) and e), i.e. a set of pre meal blood glucose measurements and a set of post meal blood glucose measurements,
5) b) , d) and c), i.e. a mean or a median value of pre meal blood glucose values, a set of pre meal blood glucose measurements and a mean or a median value of post meal blood glucose values,
6) b), d) and e), i.e. a mean or a median value of pre meal blood glucose values, a set of pre meal blood glucose measurements and a set of post meal blood glucose measurements,
7) b), d) and c) and e), i.e. a mean or a median value of pre meal blood glucose values, a set of pre meal blood glucose measurements, a mean or a median value of post meal blood glucose values and a set of post meal blood glucose measurements,
8) b), c) and e), i.e. a mean or a median value of pre meal blood glucose values, a mean or a median value of post meal blood glucose values and a set of post meal blood glucose measurements, and
9) d) , c) and e), i.e. a set of pre meal blood glucose measurements, a mean or a median value of post meal blood glucose values and a set of post meal blood glucose measurements.

The diabetic patient can determine which of said embodiments that should be shown on the display and for which meals.

As an example, in embodiment 3) the diabetic patient can see a mean or a median value of pre meal and post meal blood glucose values, thus he can se his blood sugars mean or a median values before and after a meal. Subsequently, a health care personal (a physician or a nurse, etc) can based on these values before and after a meal advice the patient about future medication of bolus insulin, e.g. if values were too high more bolus insulin should then be prescribed and vice versa.

As another example, in embodiment 5) the diabetic patient can see a mean or a median value blood glucose values and a set blood glucose measurements, both pre meal, i.e. before a meal, further the diabetic patient can see a mean or a median value of post meal blood glucose values. By means of the display of the mean or a median compared to the set blood glucose measurements, either both before or after a meal, the set indicates how value fluctuates more or less around the mean or median value. Subsequently, the health care personal can have a dialogue with the diabetic patient asking why his blood glucose levels fluctuates that much and then advice him to try to obtain a lower, a higher level (as compared to what is a suitable blood sugar level for that patient) or just a more stable blood sugar level.

Further, in the dialogue, the mean or the median value of blood glucose values after the meal could be discussed with the patient, the mean or the median value could again be compared to a suitable blood sugar level for the patient to be achieved after the meal. Consequently, an advice from the health care personal could be given securing that the patient get in compliance with his treatment regimen.

As another example, in embodiment 7) the diabetic patient could have chosen to see b), d), c) and e) at the same time for a certain meal. Thus in this embodiment of the invention, the diabetic patient can see more data items, i.e. the mean or the median value and a set of meal related blood glucose measurements before and after said meal, and thereby having the data items related to the point of time for the chosen meal. By means of the display of the mean or a median compared to the set blood glucose measurements both before or after said meal, the sets again could indicate how value, before and after the certain meal, fluctuates more or less around the mean or median value before and after the certain meal, respectively. Subsequently, the health care personal could again have a dialogue with the diabetic patient asking why his blood glucose levels fluctuate that much and were at that high level before and after the meal, if it were the cases, and then advice him to try to obtain a lower and more stable blood sugar level, e.g. suggesting him which doses of bolus insulin that should be administered and when in relation, e.g. before and after said certain meal.

It is an advantage of the invention that it can be used as a dialogue tool between the diabetic patient using the device and the health care personal. Moreover, the diabetic patient could use the displayed data as a tool for self management of his disease.

As mentioned before, in the prior art no method exists that can help in evaluation of blood glucose vis a vis the meal taken by the patient. Since the blood glucose level and the amount of insulin to be administered to the patient is directly dependent on the time and the kind of meals consumed by the patient, it is of crucial importance that consideration of this factor be taken while displaying patient's data. Once a correlation between the point of time for the meal intake and for the blood glucose level is established, it can be used to determine- the appropriate amount of insulin to be administered.

In general, any of these embodiments can be used as a dialog between the diabetic patient and his physician or nurse, further the patient himself could take actions from the shown embodiments, e.g. considering when and in which dose(s) bolus insulin should be administered for the future.

While taking insulin dosages, one of the most important factors that has to be taken into consideration is the meal intake close to the point of time of insulin administration. The amount of decrease of the glucose level that would be the consequence by insulin administration has to be carefully calculated in view of the meal taken/to be taken, time of previous meal, next meal and other related factors. A flexible and effective diabetes regime demands that food intake is to be carefully matched with appropriate amount of bolus insulin in order to reach the proper glucose level.

As discussed, a mean or median value of blood glucose values along with a set of blood glucose measurements can be displayed before and after each meal. It is thus an advantage of the present invention that it can be used display the patient's blood glucose level in relation to his meal intake This is illustrated in the following example: Example: If there is a measured high blood glucose level at 1 pm, the question that arises is: is this a high blood glucose level a high level before or after the lunch? If it is before the lunch, i.e. this high blood glucose value is then related to the breakfast, and accordingly the insulin related to the breakfast may need to be adjusted (increased), whereas if the measured high blood glucose level is after the lunch, it may instead be the insulin related to the lunch that needs to be adjusted. As can be seen if the high blood glucose level relates to the breakfast, insulin related to the breakfast may need an adjustment, conversely if said high blood glucose level instead is related to the lunch intake of food, insulin administered around lunch may need an adjustment instead.

It is thus an advantage that diabetic patient's blood glucose level is considered in relation to the point of time for the meal intake instead of the time of the day, since the time of the day and the blood glucose level not necessarily can be related to a meal.

It is a further advantage of the present invention of the invention that the patient's blood glucose level can be displayed against the three various points of times for the meals of the day, and that the use of said data may be used to help and guide the user in performing corrective actions based on blood glucose levels related to meal intakes, i.e. glucose levels related before each of the three meals and after each of the three meals. The corrective actions could be administration of insulin or an exercise.

Thus, the present invention provides for an enhanced display of diary data in which the blood glucose readings are shown versus the three meal intakes in a day. This kind of display helps in either an automatic detection of habits of the user or can also act as an aid to spot patterns in the data and take corrective actions, e.g. the glucose level two hours after a meal is a useful measure when evaluating diabetes treatment.

In an embodiment of the device, the processor is further configured to cause the display to display a mean or a median dosage values of bolus insulin administered at the habitual meal, e.g. 6, 7 and 8.4 could be displayed for corresponding two meals.

In an embodiment of the device, the processor is further configured to cause the display to display a point of time for the habitual meal and optionally information indicating the nature of the meal, e.g. 7:30, 12:30, and 18:45 corresponding three meals, the nature of the meals could be shown as breakfast, lunch and dinner, respectively.

The diabetic patient could select to have said items displayed for two or three different meals, the diabetic patient could determine for which meal(s) items are to be displayed.

In an embodiment of the device, the processor is further configured to cause the display to display a mean or a median value for bedtime glucose values.

In an embodiment of the device, said device could be a drug administration device.

In an embodiment of the device, said device could be a blood glucose measuring device.

As a prerequisite and as a background for understanding the invention, the diabetic patient using the method carried out on said drug administration device or said drug administration device will log insulin administrations and blood glucose measurement from the following actions during a day:
6:30—alarm-clock awakens
*** 6:32—blood glucose testing, before breakfast

When the patient tests his blood glucose, i.e. prick his skin for a sample of his blood for a blood glucose reading, the information may be automatically stored or manually entered to the drug administration device
**** 6:35—administration of bolus insulin, e.g. injection or inhalation of bolus insulin, around breakfast

These data is automatically stored, i.e. as the amount of insulin and the type of insulin (e.g. concentration) to the drug administration device. In a simpler drug administration device data may be manually entered
6:40—shower
6:50—breakfast
7:15—off to work
*** 8:45—blood glucose testing, after breakfast

When the patient tests his blood glucose, i.e. blood glucose reading, the information may be automatically stored or manually entered to the drug administration device
9:30—snack at a meeting
*** 11:45—blood glucose testing, before lunch

When the patient tests his blood glucose, i.e. blood glucose reading, the information may be automatically stored or manually entered to the drug administration device
**** 11:47—administration of bolus insulin, e.g. injection or inhalation of bolus insulin, around lunch

These data is automatically stored, i.e. as the amount of insulin and the type of insulin

In a simpler drug administration device data may be manually entered
12:15—lunch
****14:50—blood glucose testing, after lunch

This glucose data point is automatically transferred or manually stored to the drug administration device.
16:00—off from work
**** 18:30—blood glucose testing, before dinner

The blood glucose level may be automatically stored to the drug administration device
****18:35—administration of bolus insulin, e.g. injection or inhalation of bolus insulin, around dinner

Again, these data is automatically or manually stored, i.e. as the amount of insulin and the type of insulin (to the drug administration device
19:00—dinner
20:30—coffee
21:30—blood glucose testing, after dinner

This information may be automatically stored or manually entered to the drug administration device
**** 23:30—blood glucose testing before bedtime

This information may be automatically stored or manually entered to the drug administration device
23:35—administration of basal insulin, e.g. injection or inhalation of long acting insulin before the night

Again, these data is automatically or manually stored, i.e. as the amount of insulin and the type of insulin (long acting) to the drug administration device.

For both glucose values and insulin taken the corresponding time stamps (year, month, day, hour, minute) are to be stored with the data points.

The data items—marked with ****—are of interest prior to the use of the Modal Day and for carrying out the present invention and these items are in some way entered or wirelessly received to a data base. Subsequently, these data items can be retrieved for analysis according to the invention from said drug administration device.

Data items as above indicated with **** are logged, the logging typically takes places from more days (dates) which then comprise the users' diary data for several days.

Addressing the prandial day according to the invention is a variation of the Modal Day display. The time of meals is not known—and it is not expected that the user will be bothered to tell. The points of times for the bolus insulin administrations are applied instead of the meal times, assuming that the time between bolus insulin administration and start of the meal approximately is the same every day. That is a very reasonable assumption, assuming that the user uses the same sort of fast acting insulin as bolus insulin every day.

A patient should measure the blood glucose just before taking hid insulin before meals and 2 hours after meals. If the patient did that—which in the real word is rare—the following timeline is expected: blood glucose measurement, administration of bolus insulin, meal, 2 hours, blood glucose measurement, some hours, blood glucose measurement, administration of bolus insulin, e.g. injection of fast acting insulin, meal, 2 hours and so on. In the real world, a smaller number of blood glucose measurements are performed. That is not a problem for the invention—it just means fewer points are analysed and subsequently can be plotted.

In all cases, it is a prerequisite that said data—except for the time stamps of the three meals—as diary data from several days are available for carrying out the invention, i.e. as a method and the method carried out on said drug administration device.

The drug administration device may be a doser for injection of insulin in various concentrations, it may be in a simpler form as an electronic syringe equipped with displaying capabilities. For example U.S. Pat. No. 6,540,672, U.S. Pat. No. 6,656,114, US2002010432 and US2003032868 all disclose intelligent drug administration devices, which are hereby incorporated by reference in its entirety. The invention may as well be carried on a drug administration device in form of a pump also capable of infusing insulin in various concentrations as general known in the art.

Alternatively, the drug administration device may be an inhalation device: various inhalation devices exist that aid in depositing a liquid aerosol or dry aerosol powder into a patient's lungs. For example, U.S. Pat. No. 5,888,477 (which is hereby incorporated by reference in its entirety) discloses an inhaler with robust features that may be used for insulin delivery. U.S. Pat. No. 5,785,049 to Smith et al. (which is hereby incorporated by reference in its entirety) discloses a device suitable for powdered medication delivery.

Thus, in the present context, the term ‘drug administration device’ is taken to mean, an injector type device (such as a pen injector or a jet injector) for delivering a discrete dose of a liquid medication (possibly in the form of small drops), a medication pump for continuous delivery of a liquid medication, an inhaler, spray or the like for delivering a discrete or continuous dose of a medication in vaporized, ‘atomized’ or pulverized form. The invention may as well be implemented on an electronic device, such as a personal digital assistant, a cellular phone or on a blood glucose meter.

The present invention would now be described with reference to the accompanying figures, without limiting the scope of the invention.

### BRIEF DESCRIPTION OF THE DRAWINGS

FIGS. 1A and 1B show the modal day view of diary data,

FIG. 2 is a block diagram of a general computing device on which the invention might be practiced,

FIG. 3 shows a Gaussian influenced distribution for one event,

FIG. 4 shows a Gaussian influenced distribution for more events,

FIG. 5 shows a Mondays' actions for a diabetic patient,

FIG. 6 shows the Mondays' actions for a diabetic patient with pre and post meal glucose values,

FIG. 7 shows the Tuesdays' actions for a diabetic patient with pre and post meal glucose values including the actions from Monday,

FIG. 8 shows glucose values in relative time,

FIG. 9 shows the prandial day view of diary data,

FIG. 10 illustrates a ‘moving window’ presentation of information as per the invention,

FIG. 11 shows the prandial day view of diary data,

FIG. 12 illustrates a ‘moving window’ presentation of information as per the invention,

FIG. 13 discloses a Week view presentation,

FIGS. 14 and 15 show plots of insulin taken and BG measurements,

FIG. 16 illustrates a prandial plot,

FIG. 17 illustrates a prandial plot with a slider, and

FIG. 18 illustrates a prandial plot with highlighted values.

### DETAILED DESCRIPTION OF THE FIGURES

As mentioned earlier, in order to monitor his lifestyle, a diabetic maintains a diary in which he logs various parameters that are important for giving an insight into his living habits and present state of health. For example, apart from the obvious things like blood glucose reading, insulin administration, etc., the amount of meals/carbohydrates consumed, exercise done/calories burnt etc can also be recorded. A combination of one or more of these parameters along with their analysis helps in detecting any unwanted deviation from what is expected for a healthy life. As mentioned hereinbefore, various tools exist in the market that assist the user in entering his data, generate report and perform a subsequent analysis. Various types of reports can be generated each catering to a specific need. One such report is the modal day report as mentioned earlier. In this report, data like blood glucose, insulin etc are shown versus the time of the day for a particular period of time. This helps in detecting patterns (good or bad) in readings and deciding on corrective steps to be taken.

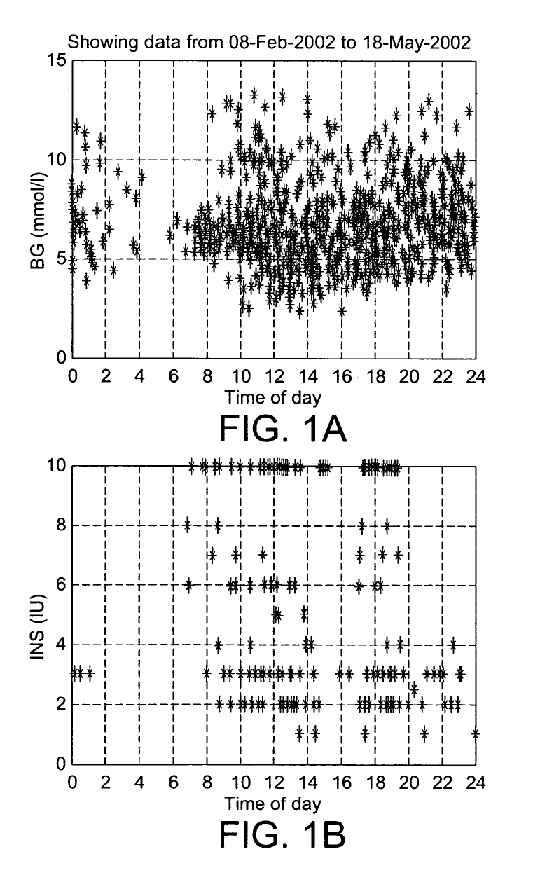

FIGS. 1A and 1B show the modal day view of diary data, i.e. they show such a modal day graph for blood glucose (in mmol/l or mM) and insulin readings for the period 8 Feb. 2002 to 18 May 2002 charted against an x-axis representing hour (time) of the day at 2 hour intervals. Such graphs and other analysis is generally a computer-implemented method.

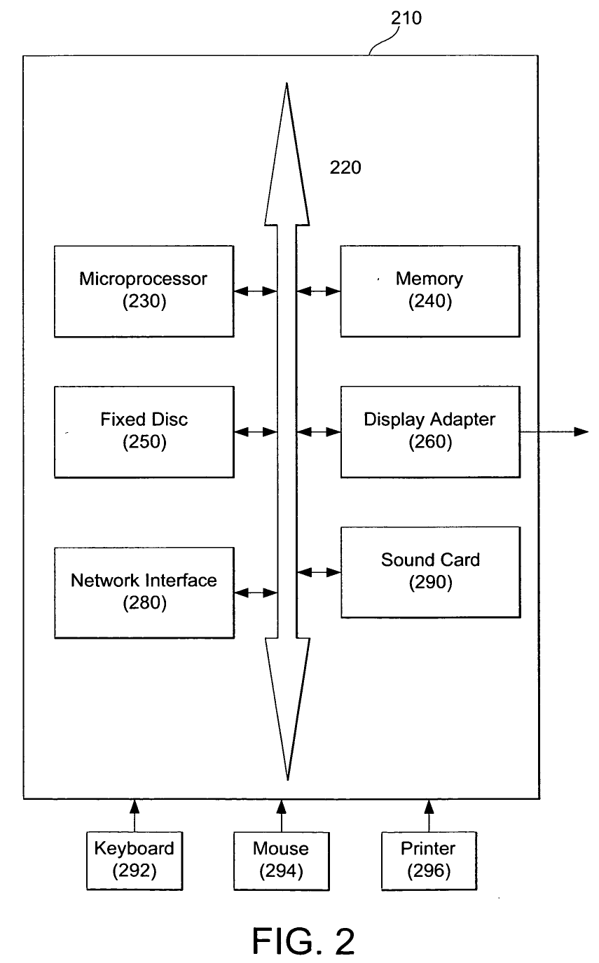

FIG. 2 is a block diagram of a general computing device on which the invention prandial day might be practiced. Said device can be a drug administration device or a blood glucose meter as well. This computer implemented method can be run on any general purpose computing device/computer system as shown in the figure, which shows its internal structure. The computer system (210), e.g. a device consists of various subsystems interconnected with the help of a system bus (220). The microprocessor (230) communicates and controls the functioning of other subsystems. Memory (240) helps the microprocessor in its functioning by storing instructions and data, e.g. diary data and determined time stamps for meals, determined pre and post meal glucose value during its execution. Fixed Drive (250) is used to hold these data, e.g. in a database structure and instructions permanent in nature like the operating system and other programs. Display adapter (260) is used as an interface between the system bus and the display device (270), which is generally a monitor or a display. In other words, the display is interfaced with said processor, where the processor can be configured to cause the display to display various data as graphics, numbers text and any combinations thereof. This display can be used to display diary data, the determined time stamps for meals, determined pre and post meal glucose value and other data of interest. The network interface (280) is used to connect the computer with other computers on a network through wired or wireless means. These devices on the network can also be drug administration devices. These drug administration devices as explained in the prior art documents are capable of storing patient related data such as drug dosage, determined time stamps of meals, blood glucose level etc. These devices communicate with the computing device using various communication mediums. The communication means can be wired or wireless such as cable, RS232, Bluetooth, infrared etc using various communication protocols such as TCP/IP, SSL etc. The computer system might also contain a sound card (290). The system is connected to various input devices like keyboard (292) and mouse (294) and output devices like printer (296). Various configurations of these subsystems are possible. It should also be noted that a system implementing the present invention might use less or more number of the subsystems than described above.

This arrangement between the drug administration device and the computing system—on both of which the invention can reside—can be as simple from a one to one link between the two. But at the same time it can also be expanded and customized as per the need to establish an efficient patient-doctor-relative-peer network. For example the computing system may periodically logon to a Local Area Network, or Internet to transmit the user readings on a remote database server that might be used to generate reports from a different computing system such as that of a doctor, relative of the patient and the like. These computing devices can be general-purpose desktops or other variations such as laptop, cell phones, PDAs, etc.

The method is incorporated in the aforementioned computing devices as by instructions in the software that are carried out by the computer system. Again, the software may be implemented as one or more modules for implementing the method steps.

In particular, the software may be stored in a computer readable medium, including the storage device or that is downloaded from a remote location via the interface and communications channel from the Internet or another network location or site. The computer system includes the computer readable medium having such software or program code recorded such that instructions of the software or the program code can be carried out. The use of the computer system preferably affects advantageous apparatuses for constructing a runtime symbol table for a computer program in accordance with the embodiments of the invention.

The computer system is provided for illustrative purposes and other configurations can be employed without departing from the scope and spirit of the invention. The foregoing is merely an example of the types of computers or computer systems with which the embodiments of the invention may be practiced. Typically, the processes of the embodiments are resident as software or a computer readable program code recorded on a hard disk drive as the computer readable medium, and read and controlled using the control module. Intermediate storage of the program code and any data including may be accomplished using the memory, possibly in concert with the storage device.

In some instances, the program may be supplied to the user encoded on a CD-ROM or a floppy disk (both generally depicted by the storage device), or alternatively could be read by the user from the network via a modem device connected to the computer. Still further, the computer system can load the software from other computer readable media. This may include magnetic tape, a ROM or integrated circuit, a magneto-optical disk, a radio or infra-red transmission channel between the computer and another device, a computer readable card such as a PCMCIA card, and the Internet and Intranets including email transmissions and information recorded on Internet sites and the like. The foregoing are merely examples of relevant computer readable media. Other computer readable media may be practiced without departing from the scope and spirit of the invention.

The executing steps can be realized in a centralized fashion in one computer system, or in a distributed fashion where different elements are spread across several interconnected computer systems. Computer program means or computer program in the present context mean any expression, in any language, code or notation, of a set of instructions intended to cause a system having an information processing capability to perform a particular function either directly or after either or both of the following: a) conversion to another language, code or notation or b) reproduction in a different material form.

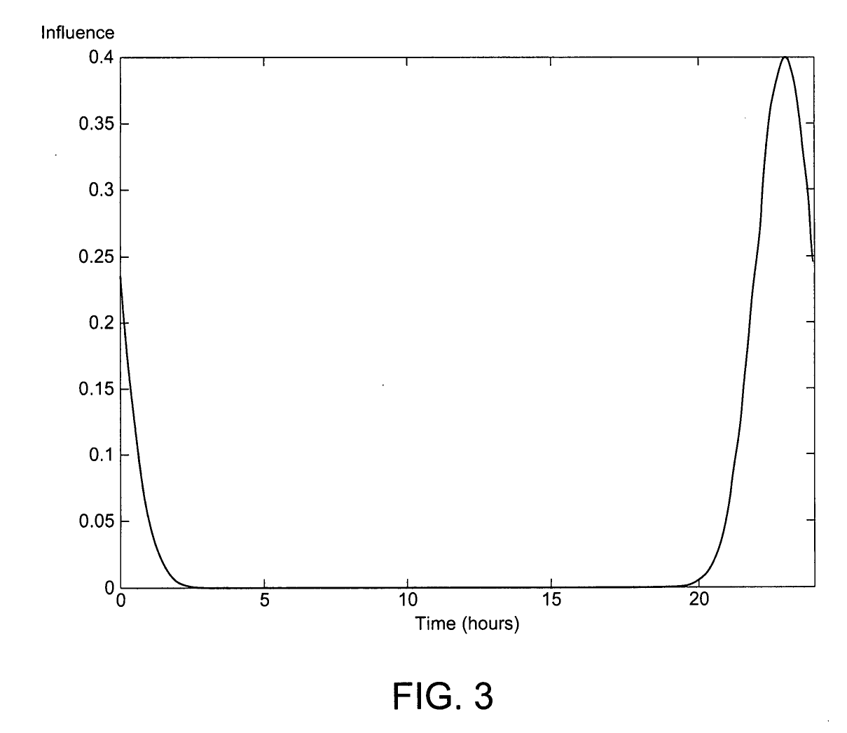

FIG. 3 shows a Gaussian influenced distribution for one event, i.e. for a meal; the first step of the prandial day algorithm is to look for habits in the form of three main meals and their insulin administrations—corresponding to the meals—located in the morning, midday and evening, specified by the algorithm as three windows in time. The next step is to filter all the instances where the time of administration falls into these windows, and for these instances, to select all the blood glucose measurements performed up to 1 hour before the time of administration of bolus insulin and up to 3 hours after the time of administration, i.e. glucose measurements before and after (pre and post) for each of said meals, respectively.

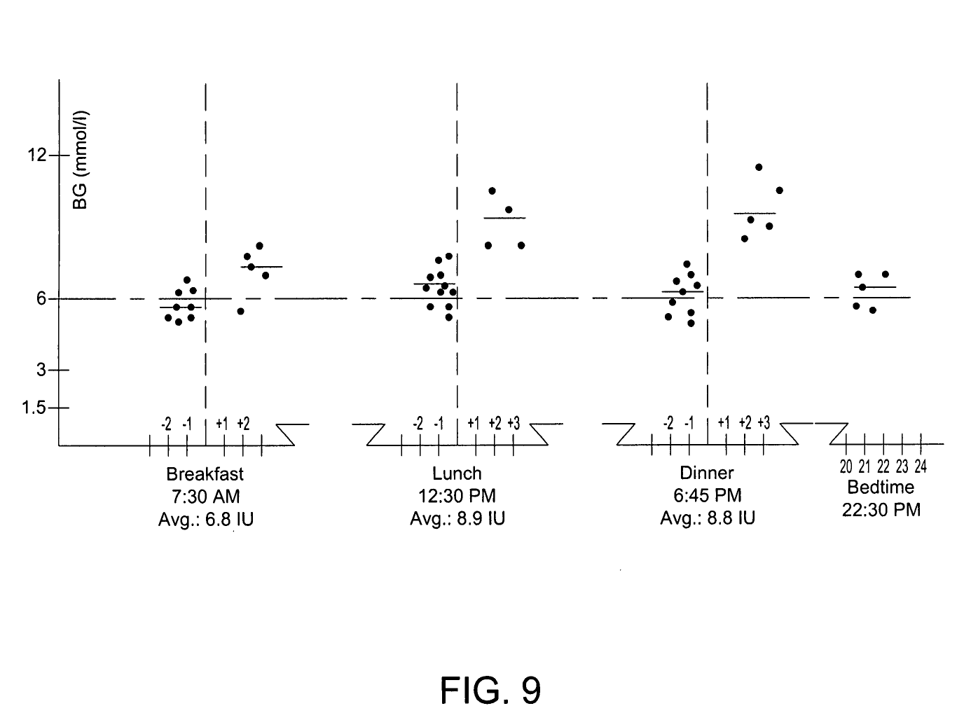

What is seen is really 4 plots combined into one: There are four time axes with time zero at the time of the dose, i.e. of bolus insulin, which is used to estimate the time of the meal. The time axes are extending from −1 to +3 hours around the meal as the user is typically supposed to measure blood glucose before insulin administrations and 2 hours after a meal. The blood glucose values from the time period in question are plotted at their time relative to the meal/time of bolus insulin administration. See FIG. 9 for such a plot.

The typical time for a meal is found the following way: typical times for meal, i.e. 8:00, 13:00 and 18:30 are guessed or by means of the three alternatives discussed in the following: Within a time window of 4 hours for each meal, a search for the real typical time for that meal is conducted. The search is performed by looking for the local maximum for a smoothed Gaussian. In this method each event is associated with a Gaussian distributed “influenced” distribution, centred at the event and with width standard deviation, which is a parameter of the algorithm. Care must be taken to ensure that time above 24 gets wrapped around to time after 0 o'clock (and similarly for time below 0). This is illustrated in the figure, which shows the influence of an event at 23 hours with standard deviation=1 hour.

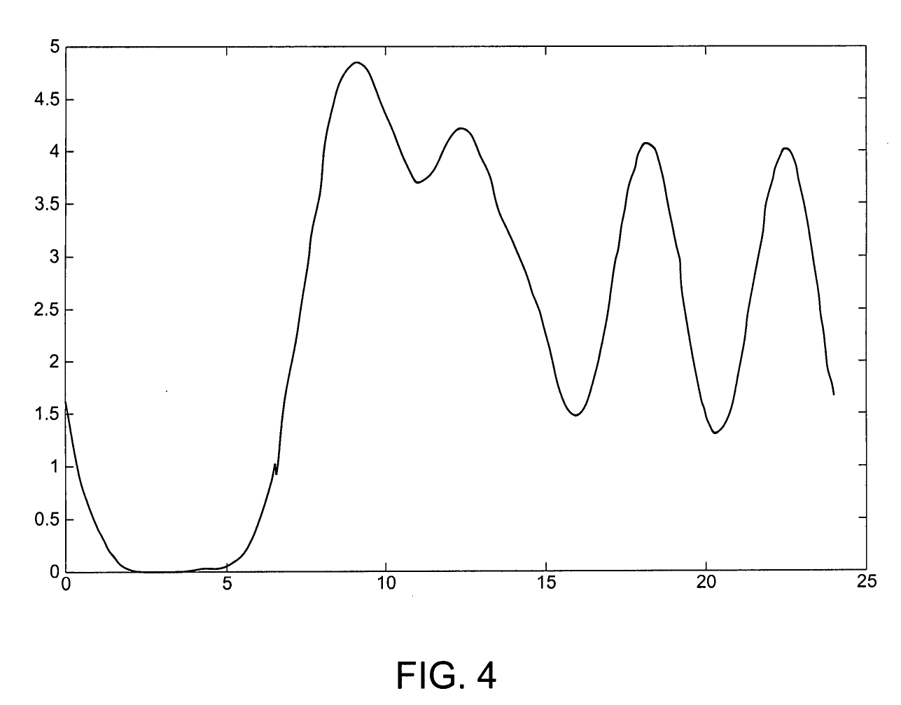

FIG. 4 shows a Gaussian influenced distribution for more events, i.e. for more meals. By accumulating the influence from all events, one obtains the curve. Four local maxima are visible. The three first are within the time windows for our guessed meal times. The points of times for these three maxima are taken to be the most typical times for corresponding three meals.

Here is the formula for the Gaussian:
ys=exp(−(ts−t)ˆ2/(2*standard deviationˆ2))/sqrt(2*pi)/standard deviation
ys: the Guassian contribution to be summed.
standard deviation: the parameter that controls the width—could be set to 1 hour.

The local maximum for each meal is indicated as the point of time for the habitual meal in the plot, see FIG. 9. That time is taken to be the most common time for meal bolus insulin administrations for that part of the prandial day plot. The median with in a predetermined time window of the points of times of bolus insulin administrations could also be computed in order to determine the point of time for the habitual meal. For data that is not Gaussian distributed the median tells what is typical, while one unusual high or low value disturbs a mean value away from the typical. Alternatively, the mean value computed with in a predetermine time window could be used to determine the point of time for the habitual meal. The bedtime part of the plot is not defined in terms of a time of bolus insulin administration, but in terms of a cluster of blood glucose, further at bedtime basal insulin typically is administered. The time of the Gaussian smoothed max is indicated below the label “bedtime”, in this case 22:50. Alternatively one could use the bed-time insulin point of time of basal insulin administration as indicator for “bedtime”.

The Gaussian smoothed max selects the most typical time—the largest cluster.

The prandial day is a way to display the blood glucose measurement data. Compared to Modal day, prandial day displays the measurements aligned to habitual meal times instead of absolute times of day.

Pseudo code for the prandial plot routine:
Detect the points of times for breakfast, lunch, dinner and bedtime habits or obtain these points of times of day from an initial setup or from default values, e.g. 8:00, 12:00 and 18:00.
For each day:
0. For each meal:
1. Look for a bolus insulin administration in a ±2 hours time window around the habitual time. If there is more than one, use the one largest. If there are two equally large doses use the one closest to the habitual time.
2. Find blood glucose measurements taken that day 1 hour before the bolus insulin administration until and including the point of time of administration. These measurements are before that given meal (i.e. before-lunch).
3. Find blood glucose measurements taken that day from (but not including) the point of time of bolus insulin administration until 3 hours after point of time for the bolus insulin administration. These measurements are after that given meal (i.e. before-lunch).
4. (If a blood glucose measurement belongs to both before one meal and after another that day, use the measurement before only.)
5. Calculate the time difference between the blood glucose measurements related to that meal that day and the bolus insulin administration that day. Plot the blood glucose measurements in the prandial plot for that meal using the relative time.
   For bedtime: Plot the blood glucose measurements in prandial plots using absolute time.
   Calculate the seven mean or median values.
   Plot the seven mean or median values, see FIG. 9.

Determine the three meals (breakfast, lunch and dinner) based on insulin administration points of times for administration of insulin, i.e. administration of breakfast bolus insulin, lunch insulin and dinner insulin, respectively. Typically, at these three occasions administration of bolus insulin is the case, whereas before bedtime basal insulin will be taken.

A way of estimating a mean value of pre meal glucose values could be implemented following pseudo code steps:
estimating pre meal glucose values for a habitual meal for one or more past days,
counting the number, n of said pre meal glucose values,
computing the mean of pre meal glucose values as the sum of these values divided by the number, n of pre meal glucose values,

A way of estimating the median value of pre meal glucose values could be implemented following the steps:
ranking said pre meal glucose values in increasing or decreasing order, and
in case the number of said pre meal glucose values, n, is an unequal number the (n/2+½) ranked pre meal glucose value is the median value of pre meal glucose values,
in case the number of said pre meal glucose values, n, is an equal number, the median value of pre meal glucose values is the mean value of (the (n/2) ranked pre meal glucose value and the (n/2+1) ranked pre meal glucose value).

Correspondingly, a procedure for the estimation of a mean and a median value of post meal glucose values could be implemented by the following steps:
estimating post meal glucose values for a habitual meal for one or more past days,
counting the number, m of said post meal glucose values,
computing the mean of post meal glucose values as the sum of these values divided by the number, m of post meal glucose values,
ranking said post meal glucose values in increasing or decreasing order,
in case the number of said post meal glucose values, m, is an unequal number, the (m/2+½) ranked post meal glucose value is the median value of post meal glucose values, and
in case the number of said post meal glucose values, m, is an equal number, the median value of post meal glucose values is the mean value of (the m/2 ranked post meal glucose value and the (m/2+1) ranked post meal glucose value).

E.g. if glucose values are 8 mM, 7 mM, 15 mM, 18 mM and 19 mM, i.e. an unequal number (5) of values, the median value of said glucose values is 15 mM as the centre or mid value. The mean value of said glucose values would be (8 mM+7 mM+15 mM+18 mM+19 mM)/5.

E.g. if post meal glucose values are 8 mM, 7 mM, 15 mM, 18 mM, 19 mM and 20 mM, i.e. an equal number (=6) of values, the median value of said post meal glucose values then is (15+18) mM/2. The mean value of said post meal glucose values would then be (8 mM+7 MM+15 mM+18 mM+19 mM+20 mM)/6.

From a large data set (from one person) the habitual meals have been calculated to be 7:30, 12:30 and 18:45. Habitual bedtime is at 23:00. The points of times for bolus insulin administration each following day change these points of times for the habitual meals slightly, but for the example in the next four figures the points of times for the habitual meals stay fixed.

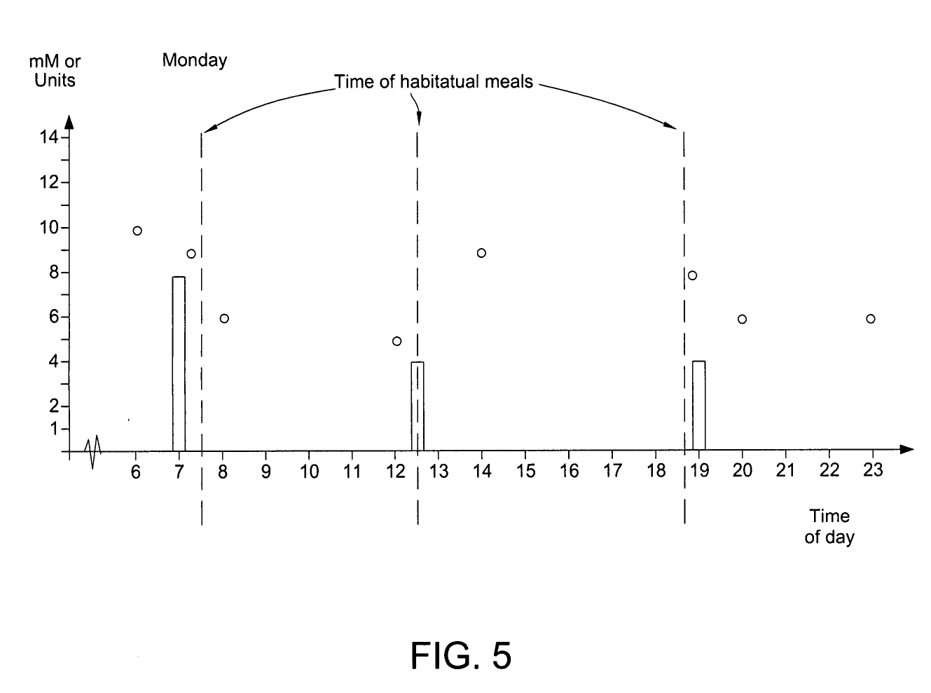

FIG. 5 shows a Mondays' actions for a diabetic patient.

On Monday the breakfast bolus insulin administration is at 7:00 (bar), lunch bolus insulin administration at 12:30 and dinner bolus insulin administration at 19:00. Blood glucose measurements are at 6:00 (10 mM), 7:45 (9 mM), 8:00 (6 mM), 12:00 (5 mM), 14:00 (9 mM), 18:45 (8 mM), 20:00 (6 mM), and 23:00 (6 mM).

The blood glucose measurements are classified as follows:

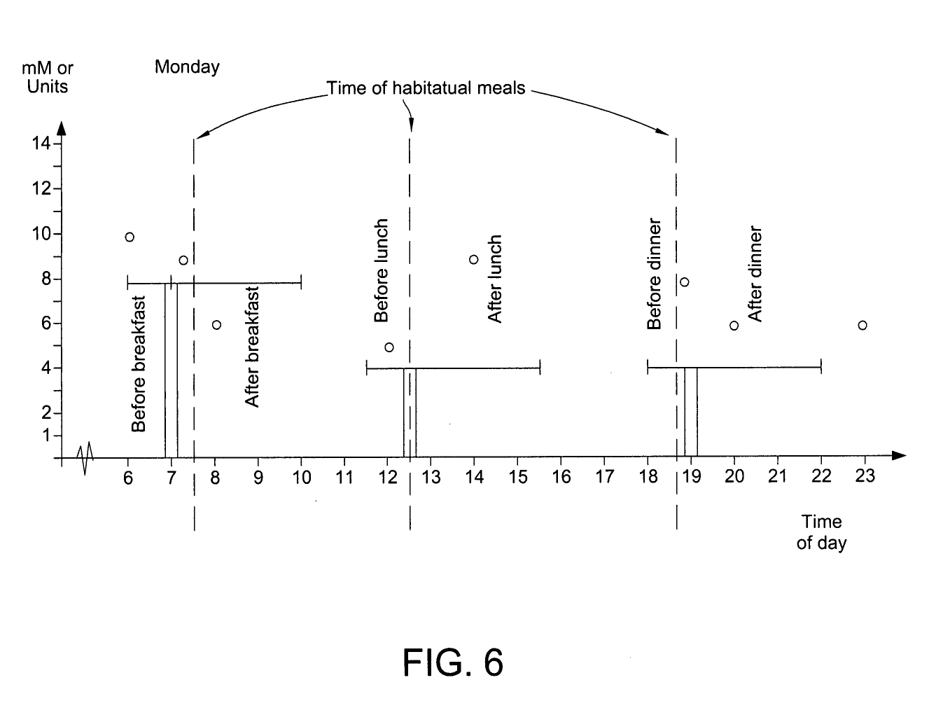

FIG. 6 shows the Mondays' actions for a diabetic patient with pre and post meal glucose values.
6:00 (10 mM)—before breakfast
7:45 (9 mM), 8:00 (6 mM)—after breakfast
12:00 (5 mM)—before lunch
14:00 (9 mM)—after lunch
18:45 (8 mM)—before dinner
20:00 (6 mM)—after dinner
23:00 (6 mM)—at bed time.

The next day is like this:

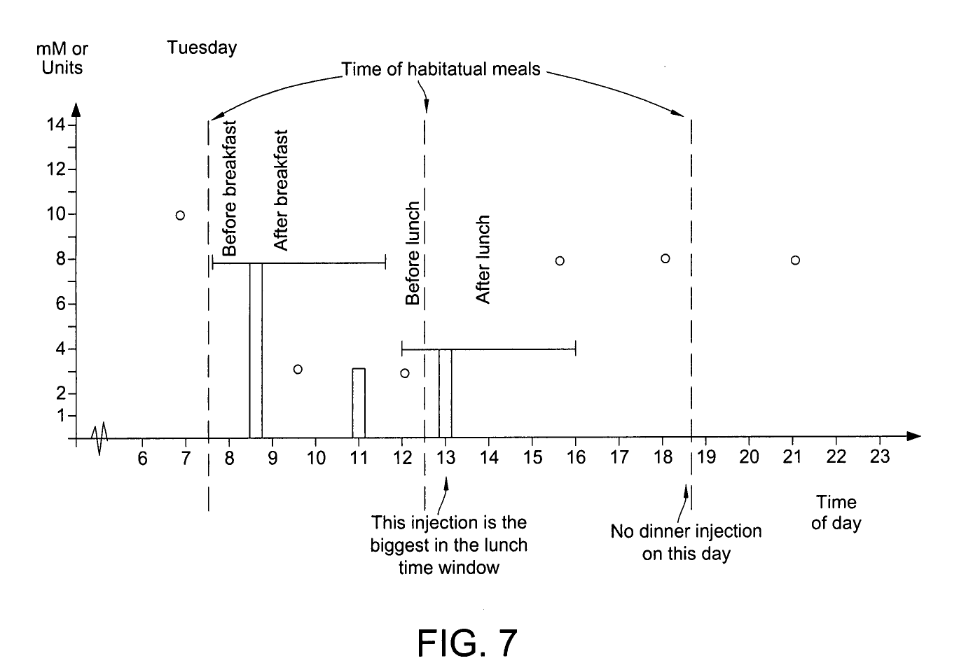

FIG. 7 shows the Tuesdays' actions for a diabetic patient with pre and post meal glucose values including the actions from Monday. Breakfast bolus insulin administration is at 8:30, so the 7:00 blood glucose measurement is outside the breakfast window for that day. Around lunch time there are two bolus insulin administrations; these are 3 units at 11:00 and 4 units at 13:00. The 4 units at 13:00 is the biggest dose administered and the other (which is lower) is therefore ignored. The blood glucose measurement at 12:00 counts as before lunch. There is no bolus insulin administration around dinner time so the blood glucose measurement at 18:00 and 21:00 is not classified.

The mean values are calculated as:
Before breakfast: 10 mM/1=10 mM
After breakfast: (9 mM+6 mM+3 mM)/3=6 mM
Before lunch: (5 mM+3 mM)/2=4 mM
After lunch: (9+8 ) mM/2=8.5 mM
Before dinner: 8 mM/1=8 mM
After dinner: 6 mM/1=6 mM.
Before bedtime: (6 mM+8 mM)/2=7 mM

Correspondingly, median values could be calculated.

The next figure shows the Prandial Day plot based on data from Monday and Tuesday:

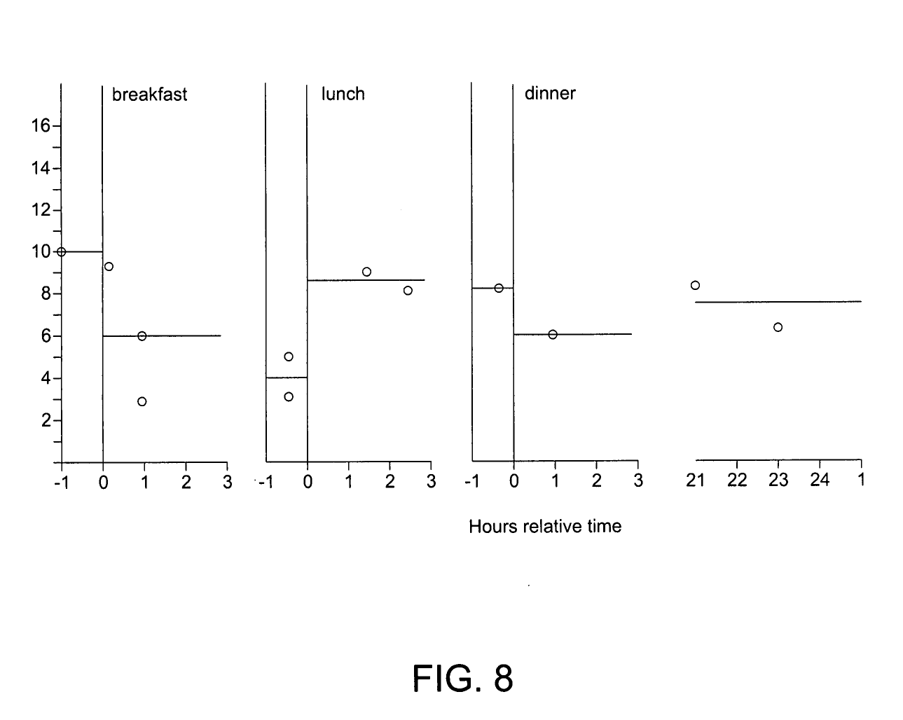

FIG. 8 shows glucose values in relative time to meal. The seven mean values are drawn as horizontal black lines. Alternatively or additionally, correspondingly seven median values could be drawn as horizontal black lines. The circles are the blood glucose measurements now shown relative the point of time of bolus insulin administration, which often is about the point of time for the corresponding habitual meal. For example the 10 mM blood glucose measurement at 6:00 on Monday is drawn at −1 hour in the breakfast plot. The 7:45 (9 mM) measurement on Monday is 0.25 hours after breakfast bolus insulin administration—even though is was performed 0.25 hour before the habitual breakfast time, i.e. the habitual breakfast meal.

It is apparent from the foregoing figures that pre meal glucose values for a habitual meal is comprised of data from one or more past days and also for data (i.e. pre meal glucose values) from an actual day.

Said data from one or more past days could be expressed as 1) a first set of data consisting the points of times of pre meal glucose measurements, each point of time relative to the point of time of bolus insulin administration and each data having a value of a pre meal glucose measurement from said one or more past days.

Whereas data from the actual day is to be identified from 2) a second set of data consisting one or more points of times of bolus insulin administrations of the patient for the actual day.

As discussed it is a prerequisite that the a) estimated point of time of the habitual meal is known.

A way of identifying pre meal glucose values from the actual day could be implemented by following the steps b) to e):
b) searching in said second set of data for one or more points of times of administration of bolus insulin in a period around a) the estimated point of time of said habitual meal on the actual day,
c) selecting a point of time for the bolus insulin administration for the actual day from b), which point of time is either the time for the largest administration if more than one administration of bolus insulin is the case, the point of time for the administration closest to said time of the habitual meal if two administrations of bolus insulin are equally large, or the point of time of administration of bolus insulin when only a single administration of bolus insulin is the case,
d) providing a set of glucose measurements for the actual day, which is one or more values of glucose measurements at corresponding one or more points of times,
e) determining one or more pre meal blood glucose values for the habitual meal each value with a corresponding time relative to the time of the bolus insulin administration from d) in a period before c) i.e. said selected point of time for the bolus insulin administration for the actual day, if any pre meal blood glucose values for the habitual meal on the actual day, whereby pre meal glucose values for the actual day now is determined.

With e) now determined, said first set of data can be updated to hold data for the past days and the actual day by means of the following step:
f) adding e) to said first set of data, whereby said first set of data now comprises pre meal glucose measurements from said one or more past days and from the actual day as well.

Referring to FIGS. 5-8, data, i.e. pre meal glucose measurements from FIGS. 5 and 6, corresponds to said a first set of data. i.e. pre meal glucose measurement from said one or more past days, in the example from Monday. Correspondingly, the determination in step d) relates to pre meal glucose measurement from Tuesday as the actual day.

Since said first set of data comprises pre meal glucose measurements from one or more past days—in the example from Monday—and from the actual day, i.e. the Tuesday as well, it is therefore possible to compute the mean value or the median value, which then can be displayed. This applies to every meal, i.e. there will be three mean values or median values, each mean value or median value related to glucose measurements before the corresponding three habitual meals.

In a preferred embodiment, said period before said selected point of time for the bolus insulin administration for the actual day is from around one hour before said selected point of time to said selected point of time.

In another preferred embodiment, said period around the estimated point of time of said habitual meal on the actual day is two hours before and two hours after said estimated time of said habitual meal.

It is therefore also apparent from the foregoing figures that post meal glucose values for a habitual meal is comprised of data from one or more past days and also for data (i.e. post meal glucose values) from the actual day.

Said data from one or more past days could be expressed as 1) a first set of data consisting the points of times of post meal glucose measurements, each point of time relative to the point of time of bolus insulin administration and each data having a value of a post meal glucose measurement from said one or more past days.

Whereas data from the actual day is to be identified from 2) a second set of data consisting one or more points of times of bolus insulin administrations of the patient for the actual day.

As already discussed it is a prerequisite that the a) estimated point of time of the habitual meal is known.

A way of identifying post meal glucose values from the actual day could be implemented by following the steps b) to e):
b) searching in said second set of data for one or more points of times of administration of bolus insulin in a period after a) the estimated point of time of said habitual meal on the actual day,
c) selecting a point of time for the bolus insulin administration for the actual day from b), which—as discussed—is the point of time is either the time for the largest administration if more than one administration of bolus insulin is the case, the point of time for the administration closest to said time of the habitual meal if two administrations of bolus insulin are equally large, or the point of time of administration of bolus insulin when only a single administration of bolus insulin is the case,
d) providing a set of glucose measurements for the actual day, which is one or more values of glucose measurements at corresponding one or more points of times,
e) determining one or more post meal blood glucose values for the habitual meal each value with a corresponding time relative to the time of the bolus insulin administration from d) in a period after c) i.e. said selected point of time for the bolus insulin administration for the actual day, if any post meal blood glucose values for the habitual meal on the actual day, whereby pre meal glucose values for the actual day now is determined.

With e) now determined, said first set of data can be updated to hold data for the past days and the actual day by means of the following step:
f) adding e) to said first set of data, whereby said first set of data now comprises post meal glucose measurements from said one or more past days and from the actual day as well.

Referring to FIGS. 5-8, data, i.e. post meal glucose measurements from FIG. 5 and 6, corresponds to said a first set of data, i.e. post meal glucose measurement from said one or more past days, in the example from Monday. Correspondingly, the determination in step d) relates to post meal glucose measurement from Tuesday as the actual day.

Since said first set of data now comprises post meal glucose measurements from one or more past days (e.g. Monday) and from the actual day (Tuesday) as well, it is possible to compute the mean value or the median value, which then can be displayed. This applies to every meal, i.e. there will be three mean values or median values, each mean value or median value related to glucose measurements after the three corresponding habitual meals.

In a preferred embodiment, said period after said selected point of time for the bolus insulin administration for the actual day is from said selected point of time to around three hours after.

In another still preferred embodiment, said period around the estimated point of time of said habitual meal on the actual day is two hours before and two hours after said estimated time of said habitual meal.

The term ‘normal distribution’ or normal distribution curve refers to a particular way in which observations will tend to pile up around a particular value rather than be spread evenly across a range of values. It is generally most applicable to continuous data and is intrinsically associated with parametric statistics (e.g. ANOVA, t tests, regression analysis). Graphically the normal distribution is best described by a ‘bell-shaped’ curve. This curve is described in terms of the point at which its height is maximum (its ‘mean’) and how wide it is (its ‘standard deviation’).

When observed frequencies (e.g. as bars) are plotted against a predicted normal distribution, it can be seen whether or not there is a rough correspondence between the two.

The simplest method of assessing normality of the distribution is to look at the frequency distribution histogram. The most important things to look at are the symmetry and peak-iness of the curve. In addition be aware of curves that indicate two or more peaks this would show a bimodal distribution and thus will not be regarded as a normal distribution.

Visual appraisals must only be used as an indication of the distribution and subsequently better methods must be used. Values of skew and kurtosis as found in Excel's Function Wizard (SKEW and KURT respectively) are another good indicator for a normal distribution, but can be over optimistic regarding the data's match with normality. Before the advent of good computers and statistical programs, users could be forgiven for trying to avoid any surplus calculations. Now that both are available and much easier to use, tests for normality (and homogeneity of variance) can be carried out as a best practice in statistics. SPSS and Minitab contain the Kolmogorov-Smirnov test, which is the principal goodness of fit test for normal and uniform data sets. Alternatively, Excel and UNISTAT provide functions to determine whether a normal distribution of data is the case.

The above tests use the same hypotheses, H's:
HO: there is no difference between the distribution of the data set and a normal distribution.
HA: there is a difference between the distribution of the data set and the a normal distribution.
The P-value will be provided by SPSS or Minitab, if below 0.05 the HO is rejected.

FIG. 9 discloses the prandial day view. Prandial day is one such variation of the modal day display. In the modal day, the blood glucose values for several days are shown versus the time of the day. The modal day has the disadvantage that one cannot see how the blood glucose values relate to meals. In the prandial day according to the invention the blood glucose values are shown at the time relative to the main meal near it.

The invention facilitates the display of the patient data in prandial format, i.e. estimation of the mean or median blood glucose levels according to a 7-point algorithm. In diabetes a 7-point algorithm, can be used for controlling the blood glucose level. The 7 points of glucose levels (mean or median) are before and after each main meal, i.e. breakfast, lunch and dinner and at bedtime. These calculated mean or median values of blood glucose levels are displayed as horizontal bold lines on the representation, which can then be compared against the doctor's defined targets for these seven points of blood glucose measurements. The figure displays corresponding values of Insulin Units injected typically around certain event in a daily routine, i.e. at breakfast, lunch, dinner and before bedtime. Correspondingly, and relating to said Insulin Units injected, the values from a blood glucose meter is plotted and shown in the prandial day view. The Prandial Day allows a detailed understanding of the pre-prandial and post-prandial blood glucose levels at standardised times. For instance the post- blood glucose, i.e. the blood glucose level 2 hours after any meal is an important measure for controlling the treatment of diabetes.

The figure shows the prandial plot for the data set. The black horizontal line indicates pre- and post meal mean or median values of glucose. The times under the first axis are the detected time of day for breakfast, lunch, dinner, and bedtime insulin, thus breakfast, lunch and dinner insulin administrations relate correspondingly to the points of times for breakfast, lunch and dinner, respectively.

The patient, doctor or any person interested can interpret the above graph so as to determine the patient's habit and their effect on his blood glucose level. Thereafter the analysis can be used to correct any wrong and/or undesired habits of the patient.

For an analysis to be carried out over a period of time, several other views can be built in which would give a better insight into the habits. Two such views are:

a) Moving Window:

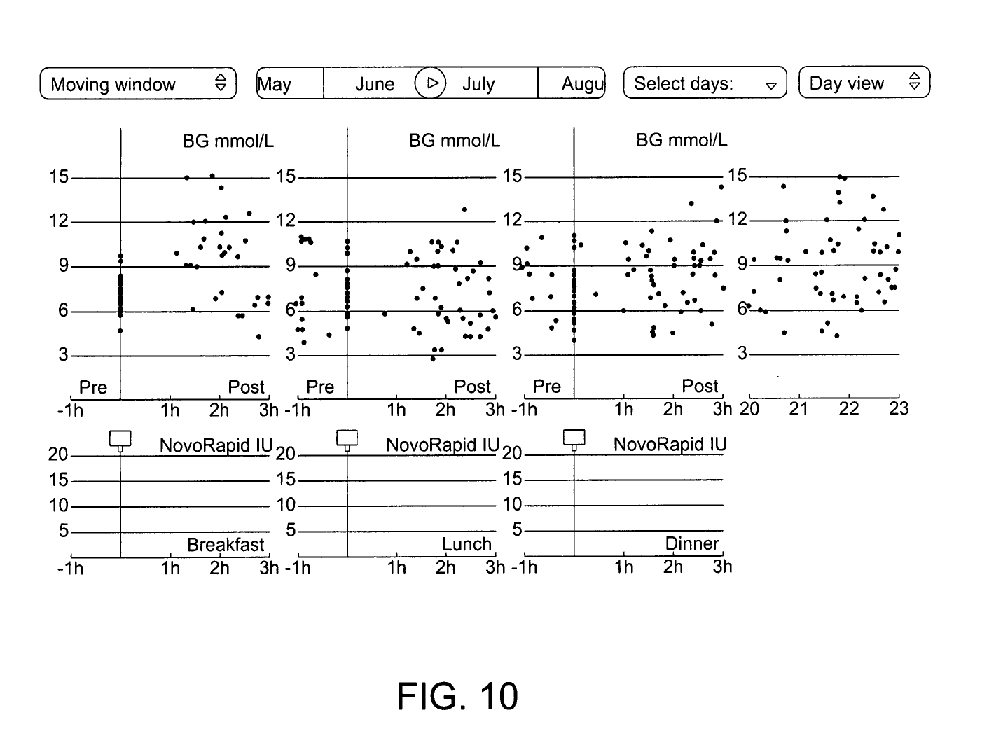

FIG. 10 shows a Moving Window representation of the patient's data. Here it is possible to select a time window to visualize data from. The default is one month in a preferred embodiment. The user can drag the time window and see the data from the time span in the Prandial day plot (white with the darker dots for blood glucose and with other dots for insulin). The user can also “press play” at the black triangle and the time window advances stepwise day by day. The data from the selected time window is drawn simultaneously in the Prandial Plot.

This invention relates to a method of displaying information to a diabetic and a medical device on which said method can be implemented and applied.

This invention relates to a so called Prandial Day. The idea is to give the user a tool to identify patterns for BG (blood glucose) and/or insulin that develop in time. For instance the user may see that (s)he tend to have a high BG Friday night but only during the summer months.

This invention relates to a medical device which records both insulin doses and blood glucose (BG) readings and their times. The device can be a doser, a BG meter, a PC, a PDA or a mobile phone.

The Prandial Day is a variation of the modal day. In the Modal Day the BG values for several days are shown versus time of the day. The Modal Day has the disadvantage than one cannot see how the BG values relate to the meals which may happen at rather different times from day to day.

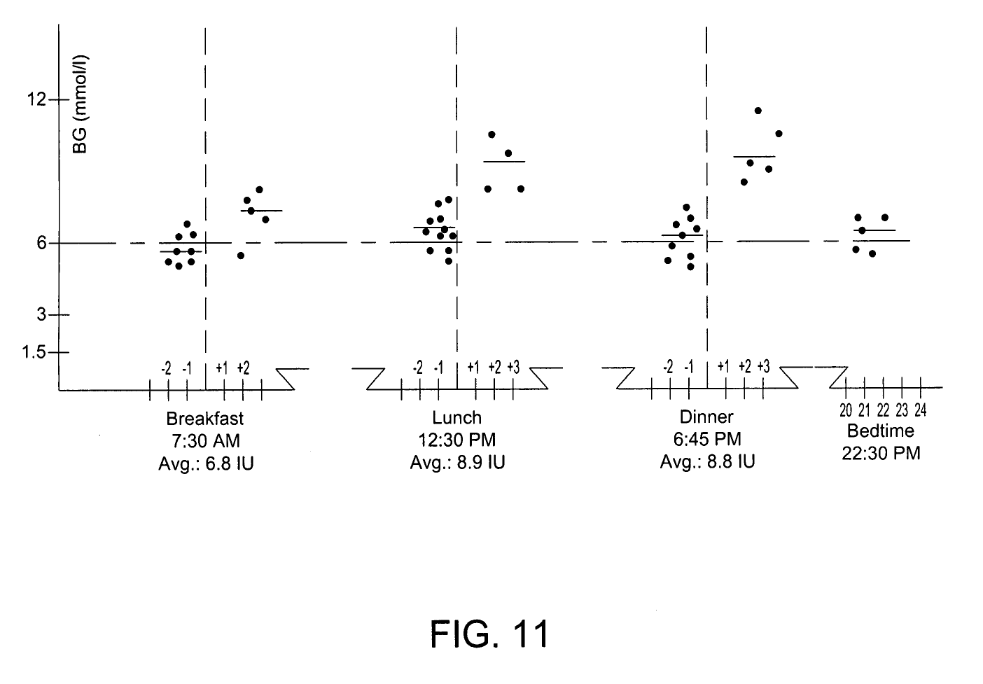

The Prandial Day is shown in the FIG. 11. Here the BG values are shown at the time relative the main meal near it. The times of the doses are used as surrogates for the meal times. The Prandial Day allows a detailed understanding of the pre-prandial and post-prandial BG levels at standardised times. For instance the post-prandial BG 2 hours after the meal is important for controlling the treatment. The invention facilitates estimate of the mean BG level according to a so-called 7-point algorithm: before and after each main meal and at bedtime, and these seven levels are displayed with horizontal bold lines on the figure. The doctor can define targets for each of these seven levels, and the Prandial Day is the preferred method for controlling this. The invention may be applied in a medical device, such as a doser, a syringe, an inhalation device, a pump all of which capable of supplying a diabetic patient with insulin in some formulation.

FIG. 11 discloses the prandial day view. The figure display corresponding values of Insulin Units injected typically around certain event in a daily routine, i.e. at breakfast, lunch, dinner and before bedtime. Correspondingly, and relating to said Insulin Units injected, the values from a blood glucose meter is plotted and shown in the prandial day view.

Said corresponding values can be entered by following pseudo code for the prandial plot routine:
Detect the breakfast, lunch, dinner and bedtime habits or get the time of day by manual entry.
For each day
   Look for a fast injection in a+2 hours time window around the habitual time. If there is more than one, use the one closest to the habitual time.
   Find the associated BG measurements in a time window of −1 to +3 hours.
   If a BG measurement belongs to both before one meal and after another, use it before only.
   For the breakfast, lunch and dinner: Calculate the time difference between the BG measurements and the fast injection. Plot the BG measurements in the three prandial plots using the relative time.
   For bedtime: Plot the BG measurements in prandial plots using absolute time.
Calculate the seven averages and plot the seven averages.

The figure shows the prandial plot for the FS data set. The black horizontal line indicates pre- and post meal averages. The times under the first axis are the detected time of day for breakfast, lunch, dinner, and bedtime insulin.

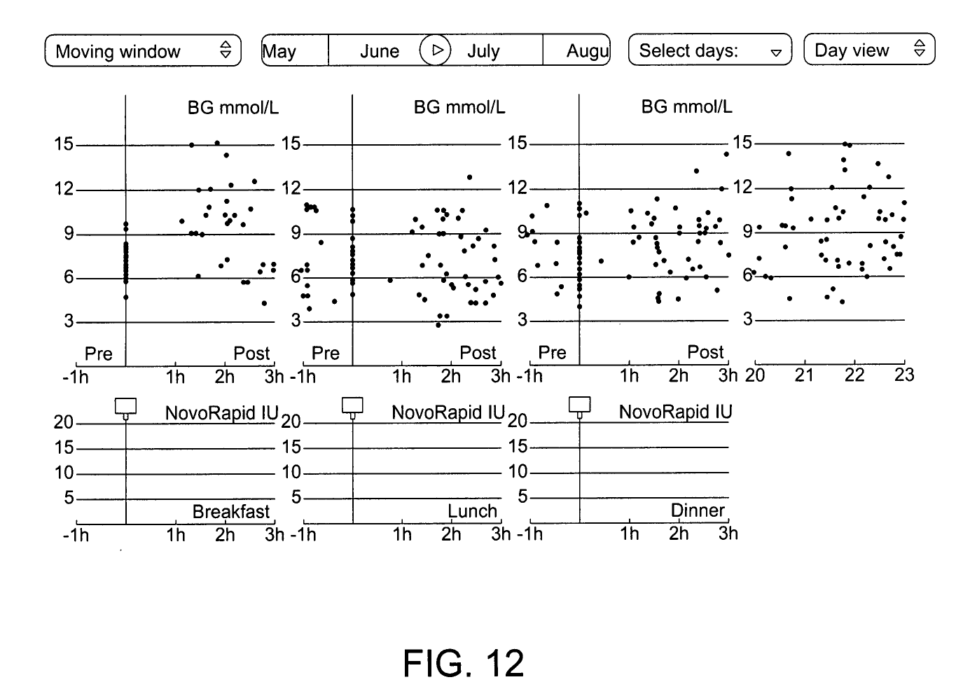

FIG. 12 discloses a Moving Window presentation. Here it is possible to select a time window to visualize data from. The default is one month. The user can drag the time window (pink) and see the data from the time span in the Prandial day plot (white with the darker dots for BG and with other dots for insulin). The user can also “press play” at the black triangle and the time window advance stepwise day by day. The data from the selected time window is drawn simultaneously in the Prandial Plot.

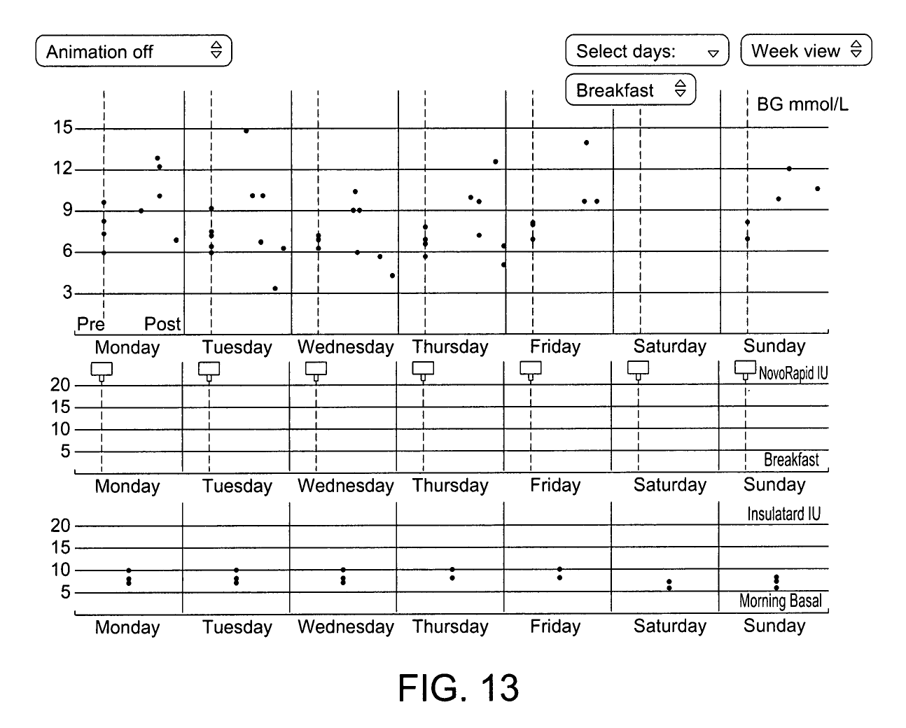

FIG. 13 discloses a Week view presentation. Week view is a variant over the basal Prandial Plot. Week view shows the breakfast, lunches or dinners of the week rather than all meal of one day. Again it is possible the run the moving window animation. Again the aim is to give the user a tool to identify patterns. The week view may for instance show (not shown in the figure) that the user tend to be low before and after Sunday breakfast but not any of the other breakfasts. (And that may suggest that the problem lies in the user's way of living Saturday rather than breakfast in general).

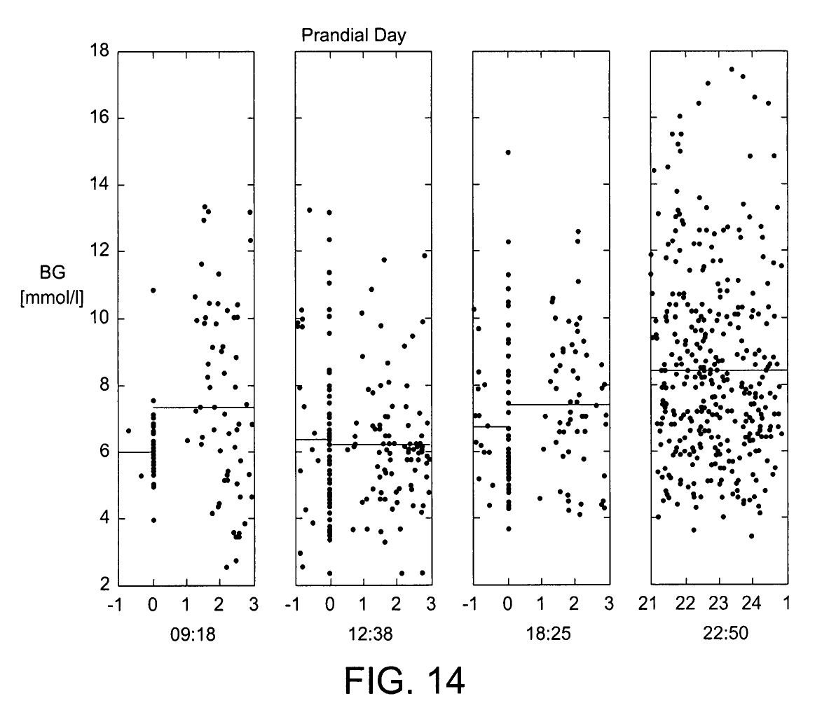

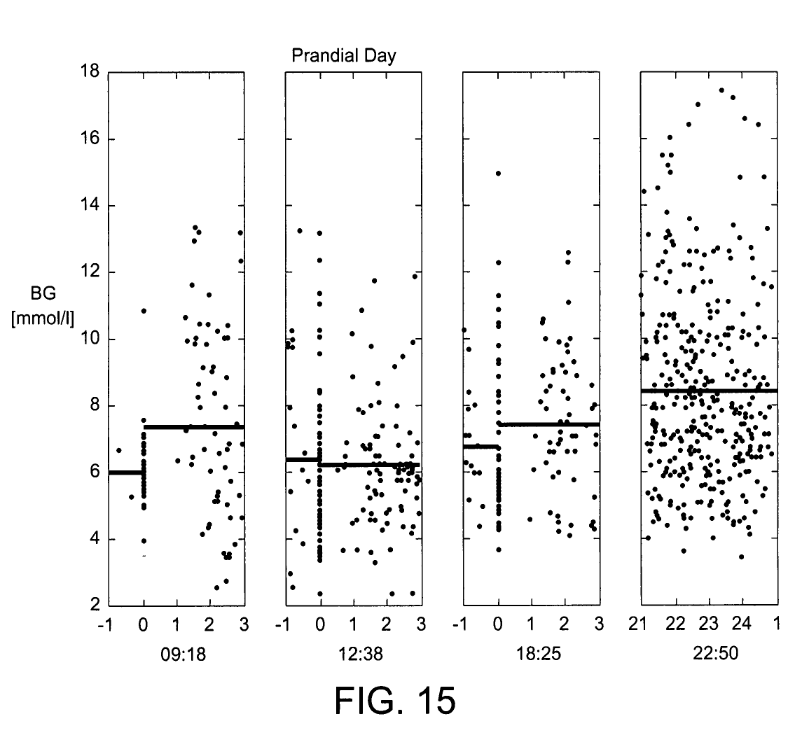

FIGS. 14 and 15 show plots of insulin taken and BG measurements, whereas the bold straight lines in FIG. 15 shows mean values of the insulin taken and the BG measurements.

In a preferred embodiment of the invention a method is implemented of displaying information to a diabetic patient, said method comprising the steps of:
logging related values of insulin injected and blood glucose readings, and
presenting said related values of insulin injected and blood glucose readings in a graphical presentation.

In a preferred embodiment of the invention a medical device is implemented, sad device comprising:
means for logging related values of insulin injected and blood glucose readings, and
means for presenting said related values of insulin injected and blood glucose readings in a graphical presentation.

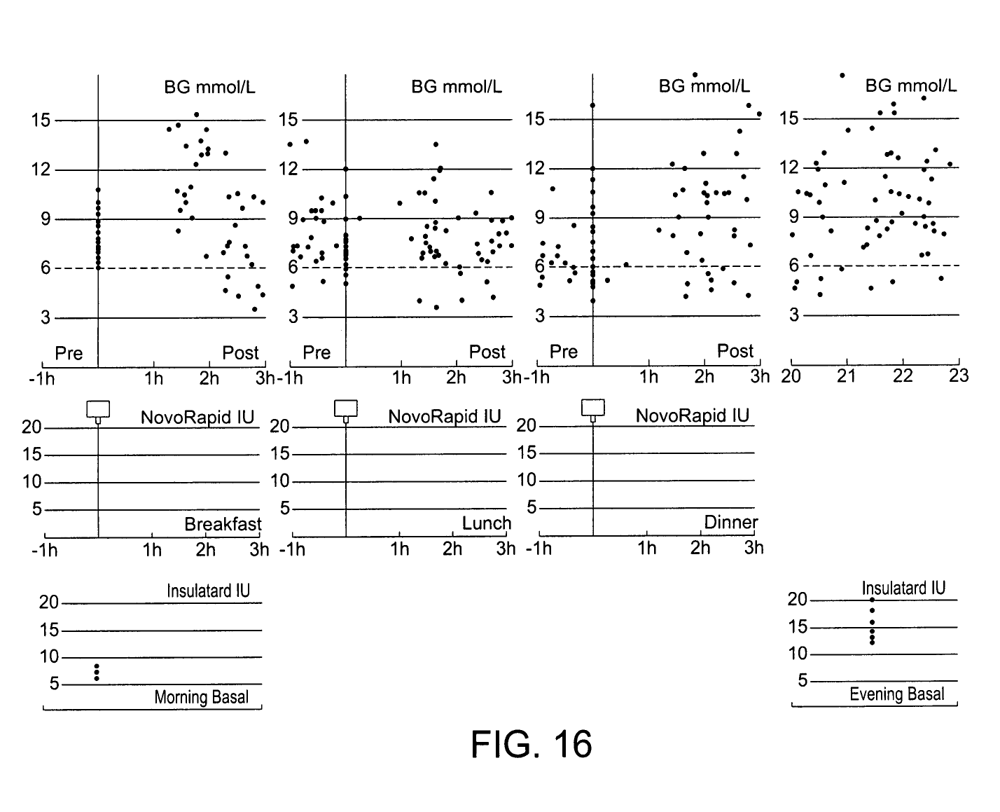

FIG. 16 illustrates a prandial plot.

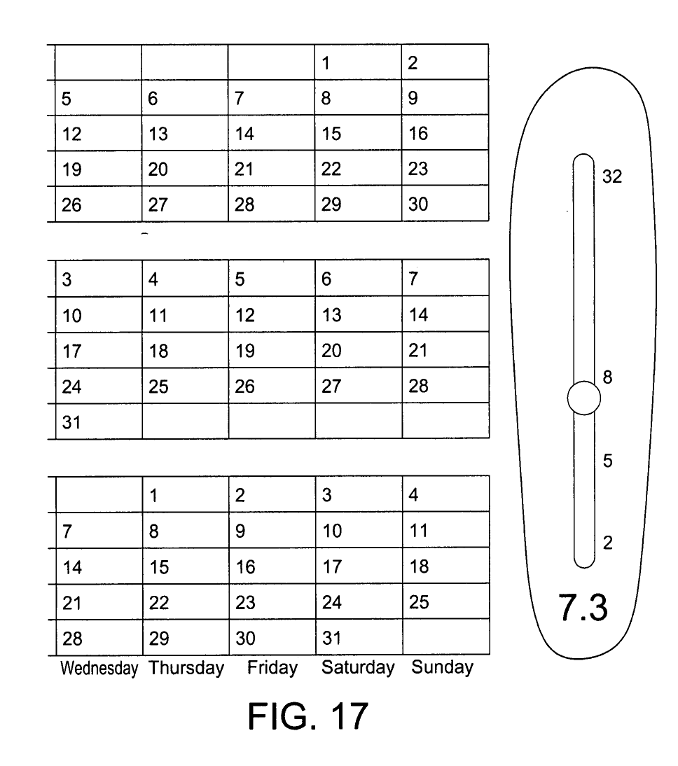

FIG. 17 illustrates a prandial plot with a slider; the diabetic patient can select to drill-down into the prandial view data by using the slider from a pattern search. The benefit is to understand what happens when the diabetic patient has a high or a low pre-prandial blood glucose values, e.g. if the patient has a high pre-prandial blood glucose value this idea would immediately present if the post-prandial values generally are high (indicating perhaps to little insulin used) or scattered from low to high, or generally low. The principle of the idea is that the diabetic patient uses the slider to select which pre-prandial blood glucose values to show in the prandial-view graphs (or in a single graph by clicking on the corresponding timeslot e.g. the breakfast view). For example if the user chooses 9.5 mmol/l (normal range e.g. 5-8 mmol/l) then only pre-prandial values ranging from 9.5 mol/l or higher are shown together with the linked post-prandial values (independently what these values post-prandial values are, and linked means the post-prandial values associated with the selected pre-prandial values, e.g. 2 hours after the selected pre-prandial view). As a generalized idea, there is one slider for each of the seven groups of blood glucose measurements: before and after breakfast, before and after lunch, before and after dinner, and at bedtime. Whenever the user selects a point on one slider (for example before lunch) only these data are shown before lunch—as before. On the six other plots only data measured the same day (as data currently shown on the before lunch plot) are shown. If the user asks: There are a lot of highs at the before lunch plot—what happens those days? The new slider-filtered plot shows exactly that.

Another possibility is just one slider and then clicking on the part of the plot to apply it to.

Yet another possibility is just one slider and the plot shows the highs of glucose measurements before any meal and the corresponding measurements after. This is simpler but the user cannot use the filter for groups of values after the meals—which may be less important.

The slider idea can also be applied to the insulin injections: Sliding up and down at the insulin slider the Prandial plot shows only measurement taken on corresponding days. For instance placing the lunch insulin slider on 7 units of basal insulin, the prandial plot shows only blood glucose measurements on days where the user took 7 units or more of basal insulin for lunch.

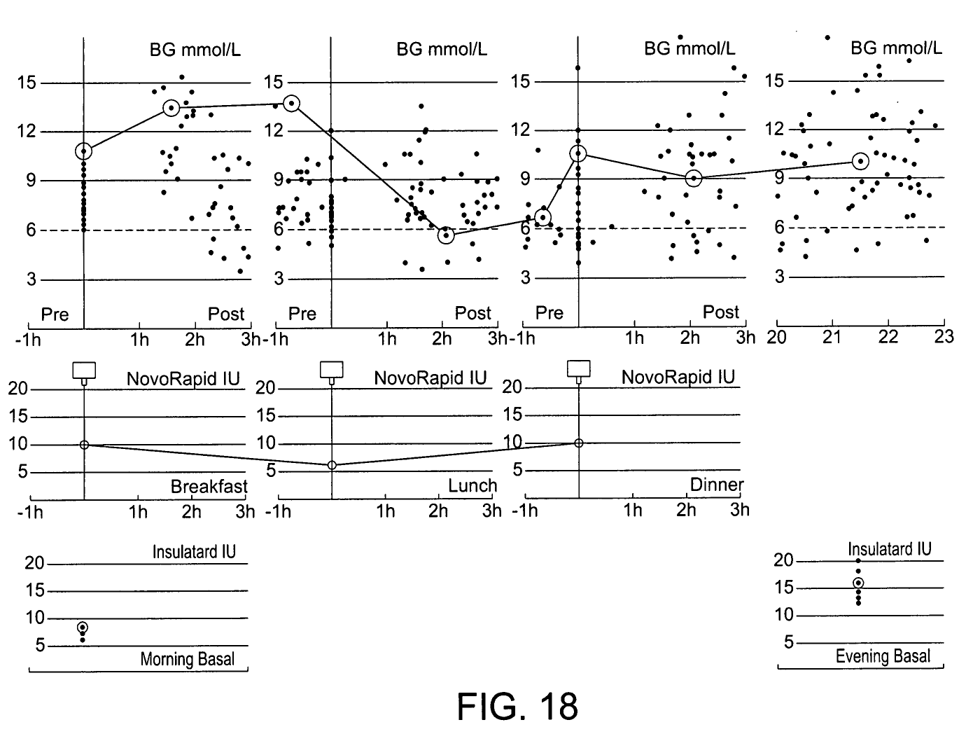

FIG. 18 illustrates a prandial plot with highlighted values, when the user clicks at a blood glucose measurement or an insulin injection value in the prandial plot the values of that day is highlighted in the same way as in the Modal plot. The user asks: Here is a high value of a blood glucose measurement—what happened that day in terms of other blood glucose measurements? The highlighting in the figure shows just how these measurements are linked for that day.

The foregoing describes only some of the various possible embodiments of the present invention, and modifications and/or changes can be made thereto without departing from the scope and spirit of the invention, the described embodiments being illustrative and not restrictive. Although the invention has been explained using diabetes as a central theme, however the invention is no way restricted to the field of diabetes. The use of a central example is to bring clarity and uniformity. The invention is equally effective in other similar application including (but once again not restricted to) general health monitoring. The aforementioned figures and their explanation are meant to be only illustrative and are uses as examples and aids to explain the invention lucidly and are in no way meant to limit the invention or take away from its essence which is hereinafter specifically stated in the following claims.

A computer readable storage medium may be a magnetic tape, an optical disc, a digital video disk (DVD), a compact disc (CD or CD-ROM), a mini-disc, a hard disk, a floppy disk, a smart card, a PCMCIA card, a ram stick, etc. or any other kind of media that provides a computer system with information regarding how instructions/commands should be executed.

All references, including publications, patent applications, and patents, cited herein are hereby incorporated by reference to the same extent as if each reference were individually and specifically indicated to be incorporated by reference and were set forth in its entirety herein.

Any combination of the above-described elements in all possible variations thereof is encompassed by the invention unless otherwise indicated herein or otherwise clearly contradicted by context.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects presented herein to the maximum extent permitted by applicable law.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).
All headings and sub-headings are used herein for convenience only and should not be construed as limiting the invention in any way.
The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.
The citation and incorporation of patent documents herein is done for convenience only and does not reflect any view of the validity, patentability, and/or enforceability of such patent documents.
This invention includes all modifications and equivalents of the subject matter recited in the claims appended hereto as permitted by applicable law.
WO 00/32088
WO 03/005891
WO 03/015838
Danish patent application PA 2004 01040
U.S. Pat. No. 6,540,672
U.S. Pat. No. 6,656,114
US2002010432
US2003032868
U.S. Pat. No. 5,888,477
U.S. Pat. No. 5,785,049

1. A device (210), comprising:
a display (270) for displaying graphics, text and/or combinations thereof,
a processor (230) that is interfaced with said display,
wherein the processor is configured to cause the display to display in a diagram, which diagram comprises a time axis indicating time relative to a habitual meal of a diabetic patient and a second axis on which units of blood glucose values are indicated, the following items:
a) an indication at the point of time of the habitual meal,
b) a mean or a median value of pre meal blood glucose values and/or d) a set of pre meal blood glucose measurements,
and
c) a mean or a median value of post meal blood glucose values, and/or e) a set of post meal blood glucose measurements.

2. A device according to claim 1, wherein the processor is further configured to cause the display to display the following item:
a mean or a median dosage value of bolus insulin administered at the habitual meal.

3. A device according to claim 1, wherein the processor is further configured to cause the display to display the following item:
a point of time for the habitual meal.

4. A device according to claim 1, wherein the processor is further configured to cause the display to display the following item:
information indicating the nature of the meal.

5. A device according to claim 1, wherein said items are displayed for two different meals.

6. A device according to claim 1, wherein said items are displayed for a day.

7. A device according to claim 1, wherein the diabetic patient determines which meal(s) to display said items for.

8. A device according to claim 1, wherein the processor is further configured to cause the display to display a mean or a median value for bedtime glucose values.

9. A device according to claim 1, wherein said device is a drug administration device.

10. A device according to claim 1, wherein said device is a blood glucose measuring device.
